(19) United States
(12) Patent Application Publication
(10) Pub. No.: US 2016/0109471 A1
(43) Pub. Date: Apr. 21, 2016

(54) LIPOPROTEIN PARTICLE NUMBER FROM MEASUREMENTS OF LIPOPROTEIN PARTICLE PHOSPHOLIPID CONCENTRATION IN LIPOPROTEIN PARTICLE MEMBRANE BILAYER

(71) Applicant: HEALTH DIAGNOSTIC LABORATORY, INC., Richmond, VA (US)

(72) Inventors: Philip GUADAGNO, Mechanicsville, VA (US); Erin Grace Summers BELLIN, Sandston, VA (US); William S. HARRIS, Sioux Falls, SD (US); Deepika DEVANUR, Richmond, VA (US); Stuart HASSARD, Richmond, VA (US)

(21) Appl. No.: 14/919,287

(22) Filed: Oct. 21, 2015

Related U.S. Application Data
(60) Provisional application No. 62/066,593, filed on Oct. 21, 2014.
(60) Provisional application No. 62/147,670, filed on Apr. 15, 2015.

(51) Int. Cl. G01N 33/92; G01N 21/64

(52) U.S. Cl. CPC G01N 33/92; G01N 21/6428; G01N 21/6402; G01N 2021/6439

(57) ABSTRACT

This application describes a method for measuring the molar concentrations of lipoprotein particles and lipoprotein subclass particles in bodily fluid by Multipixel Capillary Isotachophoresis Laser Induced Fluorescence (MPCE-ITP-LIF) and compositional analysis of spherical lipoprotein particles. The ability to measure several kinds of lipoproteins and particles in one unified system provides a useful diagnostic tool for predicting the risk of developing metabolic diseases such as cardiovascular disease and cardiodiabetes.

This application claims the benefit of U.S. Provisional Patent Application Ser. No. 62/066,593, filed Oct. 21, 2014 and U.S. Provisional Patent Application Ser. No. 62/147,670 filed Apr. 15, 2015, which are hereby incorporated by reference in their entirety.

### FIELD OF THE INVENTION

The present invention relates to systems and methods for determining the molar concentrations of lipoproteins and/or lipid particles in a biological sample. The invention also teaches a method for assessing a health risk in a subject.

### BACKGROUND OF THE INVENTION

The incidence of metabolic disorders has markedly increased in the past decade, with cardiovascular disease being the leading cause of mortality in several Westernized countries. Several studies in the art have established a correlation between the dysregulation of the levels of lipoproteins and lipoprotein subclasses and the incidence of cardiovascular disease. Accordingly, one of the clinically used methods for predicting a health risk, such as the risk of cardiovascular disease is based on determining the serum levels of cholesterol and lipoproteins.

Lipoproteins are particles in the bloodstream comprising protein moieties called apolipoproteins that are covalently or non-covalently attached to lipid particles like cholesterol as well as triglycerides and phospholipids. They are classified based on several parameters, including their density, size, and electrophoretic mobility. Lipoproteins include very low-density lipoprotein (VLDL), low-density lipoprotein (LDL), intermediate-density lipoprotein (IDL), high-density lipoprotein (HDL), chylomicrons, and lipoprotein(a) (Lp(a)) particles. The particles range in size from 10 to 1000 nm, and the particle density increases in proportion to its protein to lipid ratio. As the density of the lipoprotein increases, the size of the particle decreases. For example, LDL particles are small, approximately 26 nm particles with a density of approximately 1.04 g/mL, while HDL particles are approximately 10 nm with a density of approximately 1.12 g/mL. Each lipoprotein particle is further divided into subclasses which vary in size, density, protein, and lipid composition.

Abnormalities of lipoprotein size, for example, reduced LDL size, have been reported in diabetic patients. A predominance of large VLDL accompanied by small HDL particles is suggestive of the development of occlusive disease (the narrowing of arteries from obstructing plaques). The increase in size of Lp(a) particles in the blood indicates a risk of cardiovascular disease. Hence, it is desirable to develop technologies that permit the accurate determination of the concentrations of different kinds of lipoproteins.

A commonly used method for determining the size of lipoprotein and counting the number of lipoprotein particles is NMR. Although NMR has been routinely used for determining the size of lipoproteins like HDL, VLDL, IDL and LDL, it is ineffective for measuring certain lipoprotein classes like Lp(a). Moreover, NMR is expensive, cumbersome, and technically challenging, which can impact data accuracy. The data generated via NMR is not as accurate as that generated by other techniques such as gel electrophoresis, especially for particles Lp(a) particles.

There exist a few additional methods in the art, including ultracentrifugation, electrophoresis, and ion mobility, that are used for calculating particle numbers by first separating lipid particles based upon their physical-chemical properties, followed by the measurement of apoB particles.

A clinically acceptable method for measuring lipoproteins is gel electrophoresis. This method involves density staining of bands of apoB-containing lipoprotein particles, particularly Lp(a), VLDL, IDL, and LDL. However, this method cannot be used to measure HDL particles, and currently, there is no known method for accurately determining apolipoprotein to particle stoichiometry for HDL particles, which would otherwise offer a solution to the problem in quantifying HDL.

Density gradient ultracentrifugation has been routinely used for separating all lipid fractions. This technique involves two discrete techniques. First, the lipoprotein is separated on a gradient followed by the harvesting of the various fractions. This is followed by another set of assays for estimating the particle size. This method is laborious and time consuming, and relies on two separate methods to define lipid particles. Moreover, ultracentrifugation cannot be used to separate useful lipoproteins like Lp(a).

Another routinely used method is based on exploiting the biochemical properties of lipoproteins, and involves measuring the total cholesterol level in a given sample by conducting a biochemical assay. Measurements of total cholesterol in a given sample of isolated lipoprotein subtype are also not for determining particle size or number, however. This is because the standard laboratory methods for cholesterol measurement measure both the free cholesterol (FC) in the membrane bilayer of the lipid particle as well as the esterified cholesterols in the center of the particle. Because the esterified cholesterols in the center are mixed with triglycerides in varying proportions dependent upon a host of genetic, dietary and disease factors, total cholesterol correlates only loosely with particle sizes and is not useful for generating clinically precise and accurate data for particle numbers. Patent application WO2014145678 for particle number analysis describes one solution to this problem thru precipitation protocols for HDL and LDL subclasses. Prior to the WO2014145678 application, there was no existing technology able to measure molar concentrations of lipid particles in all spherical lipoprotein particles accurately and in a single experimental step.

Thus, there is a need in the art to develop a unified system that can be used to accurately measure molar concentrations of all spherical lipoproteins, particles, or subclass, in one single step. Likewise, there is a need for a method that is able to accurately determine both particle number of lipoproteins as well as size of lipoproteins. This invention addresses this need in the art.

### SUMMARY OF THE INVENTION

Owing to the association of lipoprotein particle number with relative health, a commercial need exists to precisely and accurately measure the amount of lipoproteins, subclass, and particles in a biological sample. Thus, one purpose of the invention is to provide a method for measuring the molar concentrations of lipoproteins and particles, including but not limited to HDL-P, LDL-P, VLDL-P, IDL-P, and Lp(a)-P, based on the phospholipid concentrations of the particles and the lipoprotein particle number of spherical lipoproteins, subclasses, and particles.

One aspect of the invention relates to a method for determining the molar concentration and/or particle number of a lipoprotein or lipid particle present in a biological sample. This method involves contacting a biological sample with a non-specific lipophilic dye under conditions suitable for the non-specific lipophilic dye to bind to the lipoprotein, or a lipid particle thereof, to form a lipophilic dye-labeled lipoprotein, wherein the biological sample comprises a signal-producing lipoprotein standard. The method further involves subjecting the dye-labeled lipoprotein to a capillary isotachophoresis laser-induced fluorescence (CE-ITP-LIF) system; detecting and comparing signals produced by the non-specific lipophilic dye and the signal-producing lipoprotein standard; and quantifying, based on said detecting and comparing, the molar concentration and/or particle number of the lipoprotein or lipid particle in the sample, wherein the detected signals are proportional to the molar concentration and/or particle number of the lipoprotein or lipid particle in the sample.

A second aspect of the invention relates to a method of assessing a health risk in an individual. This method involves determining the particle number and/or molar concentration of a lipoprotein or lipid particle n a biological sample from the subject according to the first aspect of the invention. The method further involves assessing the health risk of the subject based on the particle number and/or molar concentration of the lipoprotein or lipid particle.

A third aspect of the invention relates to a system for assessing the quantities of a spherical lipoprotein particle or a lipoprotein particle subclass in a bodily fluid. The system comprises a separation apparatus for isolating the spherical lipoprotein particle or lipoprotein subclass from the non-lipoprotein components in the biological sample; a detector for detecting a signal indicating the presence of lipoprotein particle and phospholipid; a module for converting the amount of phospholipid measured to an output value that is indicative of the risk of developing the metabolic disorder. The system can comprises a storage module for the output value thus obtained, and a module for generating a report based on output value for the patient's health care provider.

The system and methods described herein provide for the simultaneous separation and detection of lipoprotein or lipid particles present in a biological sample. The advantages of the methods and system of the present invention include: the direct characterization of a complete lipoprotein profile including subclasses, Lp(a)-P and chylomicrons; measuring lipoproteins reported in molar concentrations, nmol/L, conventionally known as Particle number, PN; the complete automation for high sample thru-put or individual/manual protocols for low sample thru-put; and cost and labor efficient and uncomplicated relative to NMR cost and operational protocols. The methods of the present invention are fast, reliable, accurate, and can be automated and used for high-throughput sample analysis.

### BRIEF DESCRIPTION OF THE DRAWINGS

FIG. 1 is a schematic drawing of a system comprising two optics zones. Optics zone 1 comprises an optical rail on which are arranged a 445 nm or other specific wavelength laser or laser diode. Light form these sources is focused through a series of optical components comprising, but not limited to, a line generator, a crossed linear polarizer, and a neutral density filter. Light from Optics zone 1 is focused onto a 12.5 mm area of a 100 μM internal diameter fused silica capillary (˜365 μM o.d.) in which a 20 mm viewing window has been created by the thermal removal of the polyamide sheath. The light then passes through the sample that is being separated by ITP and excites the fluorescent label attached to each analyte molecule. Emitted light energy, at a wavelength specific to the fluorescent label is then focused to a 512 pixel photo diode array (“PDA”) through another series of optical component called Optics zone 2. Optics zone 2 comprises a set of imaging lenses (e.g., convex lenses), and an orthogonal crossed linear polarizer. After passing through a cut-on filter that transmits above a certain wavelength, the light energy reaches the detector where the data is acquired on the PDA and the signal is processed by proprietary signal processing algorithms.

FIG. 2 is a schematic drawing of a system comprising two optics zones. Optics zone 1 comprises a 445 nm LED/Laser/Laser Diode. Optics zone 2 comprises an off axis concave diffusion grating that focuses wavelength dispersed achromatic light of a wavelength specific to the fluorescent label onto the 512 pixel photo diode array. By rotating the diffraction grating, the light energy reaches the detector where the data is acquired on the PDA and the signal is processed by proprietary signal processing algorithms. An additional cut-on filter or crossed polarizer may be added.

FIG. 3 is a schematic drawing of a system comprising a simple off axis translucent parabolic mirror.

FIG. 4 is a schematic drawing of a system comprising two optics zones. Optics zone 2 comprises a fibre-optic plate (“FOP”) or coherent fibre bundle allowing proximity focusing via a cut-on filter without needing the PDA to touch the capillary.

FIGS. 5A-5C show the analysis of collected pixel data. FIG. 5A is a typical electropherogram from a single pixel of the PDA used to build up the Equiphase map shown in FIG. 5B. Each point of the Equiphase map represents a detected peak in space (pixel) and time (scan count). Tracking is performed to group sets of peaks into signal tracks, which travel in a straight line across the Equiphase map. FIG. 5C shows the fitting of such tracks with linear functions to give their velocities. Each black line in FIG. 5C represents a signal track; the gradient of the lines gives the velocity.

FIGS. 6A-6C are electropherograms of multiple samples showing the detection of individual fractions by CE-ITP-ILF. FIG. 6A shows an electropherogram of a control sample comprising CF in the absence of a biological sample. FIG. 6B shows the lipoprotein profile of several replicate biological samples prepared from patient 8 and spiked with CF. FIG. 6C shows that the lipid profile detected remains constant even after CF has degraded.

FIGS. 7A-7C are electropherograms of native samples from patient 8 prepared in the presence or absence of a lipoprotein spike. FIG. 7A shows an electropherogram of a native sample of patient 8 incubated with an HDL spike. FIG. 7B shows an electropherogram of a native sample of patient 8 incubated with an LDL spike. FIG. 7C shows an electropherogram of a HDL/VDL/LDL mixture from patient 8 incubated with a VLDL spike. The arrow indicated the possible location of the VDL peak.

FIGS. 8A-8E are electropherograms showing the lipid profiles of various biological samples. FIG. 8A shows an alignment of electropherograms from samples prepared from LDL Patient 6 (top) and LDL Patient 4 (bottom). Gel images (not shown) indicate that the LDL 6 sample contains Lp(a) and that the LDL 4 sample does not. FIG. 8B shows the alignment of electropherograms from samples prepared from patients 1-6. Samples from Patients 1, 2, and 6 should contain Lp(a). Arrows indicate possible extra peaks which could indicate the presence of Lp(a). FIG. 8C shows 3 replicate electropherograms of the HDL sample from patient 6. FIG. 8D shows the alignment and normalization of electropherograms from HDL samples of patients 1-6. Electropherograms were normalized around the CF peak (arrow). FIG. 8E shows the alignment and reproducibility triplicate electropherograms from native samples.

FIGS. 9A-9G show the lipid profiles of 6 biological samples. FIGS. 9A-9F show electropherograms of biological samples from patients 1-6, respectively. FIG. 9G shows an alignment of the electropherograms collected for biological samples from patients 1-6, normalized around the CF peak. Corrected peak areas are shown in the figure. Arrows indicate peaks corresponding to CF and LDL peaks.

### DETAILED DESCRIPTION

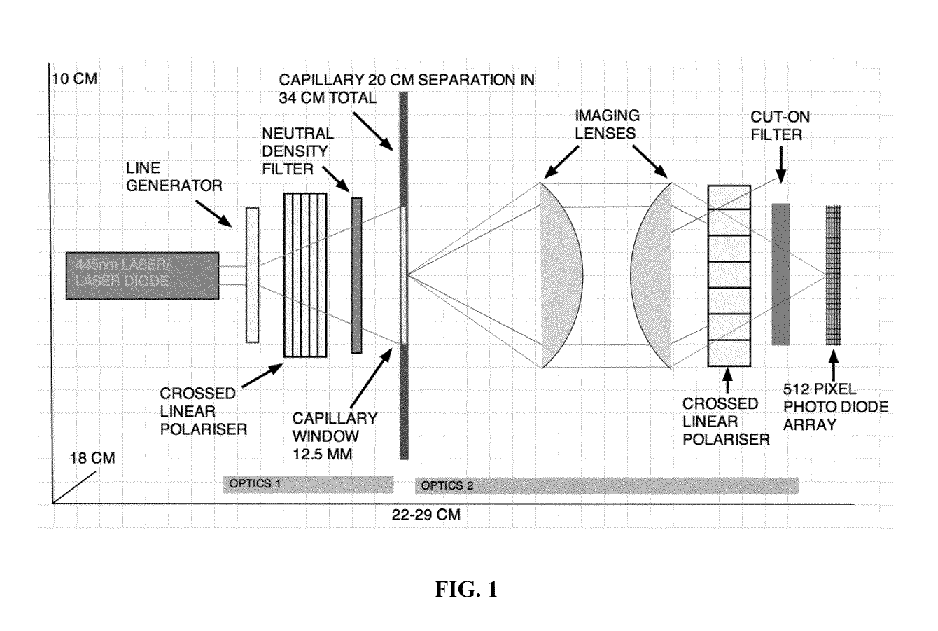

The present invention is directed to a method and system for determining the molar concentration and/or particle number of a lipoprotein or lipid particle present in a biological sample. The invention also teaches a method for assessing a health risk in a subject. These methods use a CE-ITP-LIF apparatus, which provides for the simultaneous resolution and detection of labeled lipoproteins or lipid particles present in a biological sample (see FIGS. 1-4 and Example 1).

One aspect of the invention relates to a method for determining the molar concentration and/or particle number of a lipoprotein or lipid particle present in a biological sample. This method involves contacting a biological sample with a non-specific lipophilic dye under conditions suitable for the non-specific lipophilic dye to bind to the lipoprotein, or a lipid particle thereof, to form a lipophilic dye-labeled lipoprotein, wherein the biological sample comprises a signal-producing lipoprotein standard. The method further involves subjecting the dye-labeled lipoprotein to a capillary isotachophoresis laser-induced fluorescence (CE-ITP-LIF) system; detecting and comparing signals produced by the non-specific lipophilic dye and the signal-producing lipoprotein standard; and quantifying, based on said detecting and comparing, the molar concentration and/or particle number of the lipoprotein or lipid particle in the sample, wherein the detected signals are proportional to the molar concentration and/or particle number of the lipoprotein or lipid particle in the sample.

The term “lipoprotein particle” refers to a particle that contains both protein and lipid. Examples of lipoprotein particles are described in more detail below.

The terms “particle number” or “molar concentration” as used herein refer to the number of particles present in a unit volume of a biological sample. Particle number (PN) may be in units of nmol/L.

The term “apolipoprotein” as used herein refers to a protein that combines with lipids to form a lipoprotein particle. Examples of apolipoprotein types are described in more detail below. The unique nature of the apolipoprotein is their stoichiometric relationship to lipoprotein particles, providing an estimate of the lipoprotein particle number, which is described in more detail below.

Lipoproteins are biological assemblies comprising an outer layer of protein and phospholipids and a core of neutral lipids including cholesterol esters and triacylglycerols. Lipoproteins include very low-density lipoprotein (VLDL), low-density lipoprotein (LDL), intermediate-density lipoprotein (IDL), high-density lipoprotein (HDL), chylomicron, lipoprotein X, and lipoprotein(a) (Lp(a)) particles. Each lipoprotein particle is further divided into subpopulations, which vary in size, density, protein, and lipid composition. These subpopulations of lipoprotein classes can be referred to as subclasses, subspecies, or subfractions.

Suitable biological samples according to the invention include, without limitation, fresh blood or stored blood or blood fractions. The sample may be a blood sample expressly obtained for the assays of this invention or a blood sample obtained for another purpose which can be subsampled for use in accordance with the methods described herein. For instance, the biological sample may be whole blood. Whole blood may be obtained from the subject using standard clinical procedures. The biological sample may also be plasma. Plasma may be obtained from whole blood samples by centrifugation of anti-coagulated blood. The biological sample may also be serum.

Additional exemplary biological samples include, without limitation, human biological matrices, plasma, serum, blood component, synovial fluid, ascitic fluid, and human lipoprotein fractions. The lipid fraction may be substantially pure such that it comprises a single lipoprotein and/or lipid particle class or subclass. Alternatively, the lipid fraction may be unpurified and comprise one or more lipoprotein and/or lipid particle classes or subclasses.

In one embodiment, the lipoprotein or lipid particle present in a biological sample is selected from the group consisting of very low-density lipoprotein (VLDL), low-density lipoprotein (LDL), intermediate-density lipoprotein (IDL), high-density lipoprotein (HDL), chylomicron, lipoprotein X, lipoprotein(a), and subforms and mixtures thereof.

As described herein, signal-producing lipoprotein standards may comprise one or more purified lipoprotein components or lipoprotein fractions. Alternatively, the lipoprotein standard may comprise an unpurified, but otherwise known characterized solution. Likewise, the lipoprotein standard may comprise a previously characterized biological sample.

In one embodiment, the signal-producing lipoprotein standards comprise a standard lipoprotein or lipid particle with a known concentration, a known radius, a known lipid concentration, a known lipid distribution, or a combination thereof.

According to the methods of the present invention, biological samples and/or lipoprotein standards are contacted with a non-specific lipophilic dye under conditions suitable for the non-specific lipid dye to bind to the lipoprotein or lipid particle thereof, to form a lipophilic dye-labeled lipoprotein. Suitable non-specific lipophilic dyes include fluorescently-tagged lipid anchors (e.g., fluorescently-labeled fatty acid analogs). Such optically-active components may be broadly termed lipophilic dyes, with or without the lipid anchor. An example of labeled fatty acid analog is NDB-ceramide. The NDB moiety is a useful label in the hydrophobic environment of a lipid membrane, as it has drastically different optical properties than its properties in an aqueous environment outside the lipid particle and lipid membrane. Other possible fluorescent label-linked fatty acids include ADIFAB fatty acid indicators, phospholipids with BODIPY dye-labeled acyl chains such as BODIPY glycerophospholipids, phospholipid with DPH-labeled acyl chain, phospholipids with NBD-labeled acyl chains, phospholipids with pyrene-labeled acyl chains, phospholipids with a fluorescent or biotinylated head group, LipidTOX phospholipid and neutral lipid stains. Many such options are provided by Life Technologies™ for research and production laboratory assays.

Other exemplary non-specific lipophilic dyes include, without limitation, carboxyfluorescein, BODIPY dyes, or the Alexa Fluor™ series. Such dyes are known by those skilled in the art and may be chosen from a group including, but not limited to lipophilic versions of fluorescent dyes including Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, BODIPY® FL, Coumarin, Cy®3, Cy®5, Fluorescein (FITC), Oregon Green®, Pacific Blue™, Pacific Green™, Pacific Orange™, Tetramethylrhodamine (TRITC), Texas Red®, DNA stains, DAPI, Propidium Iodide, SYTO® 9, SYTOX® Green, TO-PRO®-3, Qdot® probes, Qdot® 525, Qdot® 565, Qdot® 605, Qdot® 655, Qdot® 705, Qdot® 800, other lipophilic fluorescein derivatives such as carboxyfluorescein, carbocyanine derivatives such as iD (DiIC18[5]), DiI (or DiIC18[3]), DiI in vegetable oil, Dilinoleyl DiI, Dilinoleyl DiO, DiO (or DiOC18[3]), DiOC14(3), hydroxyethanesulfonate, DiOC16(3), DiR (DiIC18[7]), DiSC2(5), DODC (DiOC2(5)), Neuro-DiI, Neuro-DiI in vegetable oil, Neuro-DiO, Neuro-DiO in vegetable oil.

When using a lipid anchor, a variety of options may be chosen from the group including, but not limited to fatty acids, phospholipids, acyl chains such as glycerophospholipids, and neutral lipids.

Contacting the lipophilic dyes with an uncharacterized biological sample comprising lipoprotein and/or lipid particles is done to saturation of the lipid particle membranes. In addition to time, mixing and heating and cooling steps may facilitate rapid saturation of the label in the membrane.

As described in more detail herein, the phospholipid content of lipoprotein and/or lipid particles can be measured in a direct or indirect manner, through separation and precipitation or fluorescent labeling, respectively. In a separation and precipitation protocol, a capillary isotachophoresis (“CE”) system may be used to draw lipoprotein types into sharply distinguished regions in the capillary. Those distinct regions contain a type of lipoprotein corresponding to a unique electrophoretic mobility. Fractions comprising the distinct regions can be captured after separation and their phospholipid composition quantified via the methods described in US WO2014145678.

CE encompasses a family of related separation techniques that use narrow-bore fused-silica capillaries to separate a complex array of large and small molecules. High electric field strengths are used to separate molecules based on differences in charge, size and hydrophobicity. Sample introduction is accomplished by immersing the end of the capillary into a sample vial and applying pressure, vacuum or voltage. Depending on the types of capillary and electrolytes used, the technology of CE can be segmented into several separation techniques. Exemplary CE techniques include isoelectric focusing, isotachophoresis (ITA), and capillary zone electrophoresis, also known as free-solution capillary electrophoresis. Separation of lipoproteins by capillary electrophoresis is an effective technique for accurately detecting the lipid particles and relative subfractions. These methods are limited by the absence of effective and scalable methods to calculate lipid particle concentration.

CE-ITP is an electrophoretic technique in which sample ions are separated under an electric field across a length of tubing or capillary. A liquid plug comprising a biological sample to be separated is bounded by a leading buffer on one end and a trailing buffer on the other end. The leading and trailing buffers maintain the sample between them, enhancing the separations resolution. As samples migrate through the capillary, the sample components focus into bands based on their unique electrophoretic mobilities. Such bands can be distinguished by various techniques including UV light absorption, native fluorescence directly in the capillary or after elution from the capillary by subsequent gel or immunological detection.

CE-ITP has been used to separate plasma lipoproteins in preparation for subsequent analysis on a gradient gel (see Bottcher et al., “Automated Free-Solution Isotachophoresis: Instrumentation and Fractionation of Human Serum Proteins,” Electrophoresis. 19(7): 1110-6 (1998) and Bottcher et al., “Preparative Free-Solution Isotachophoresis for Separation of Human Plasma Lipoproteins: Apolipoprotein and Lipid Composition of HDL Subfractions,” J Lipid Res. 41(6): 905-15 (2000)). In particular, Bottcher describes that sample components were separated from one another through the use of spacers. Analysis required use of a transfer gel, gradient gel electrophoresis and western blotting for detection. This and other current capillary isotachophoresis methods do not permit the quantification of the molar quantities of lipoproteins present in a biological sample, which is a more accurate predictor of the levels of lipoprotein subparticles, and the risk of developing a disease.

The methods of the present invention utilize a capillary isotachophoresis laser induced fluorescence system (CE-ITP-LIF) to separate the lipoprotein particles based on their electrophoretic mobilities and to detect the signal produced by the labeled phospholipids in a biological sample. The lipoprotein and/or lipid particles are separated into a spectrum of bands comprising similar molecules.

In one embodiment, the CE-ITP-LIF system separates the components of the sample from one another along a common capillary.

In another embodiment, the CE-ITP-LIF system is a multiplex capillary isotachophoresis laser induced fluorescence (MPCE-ITP-LIF) system. MPCE-ITP-LIF systems comprises a parallel array of capillaries to simultaneously separate multiple samples.

The CE-ITP-LIF and/or MPCE-ITP-LIF systems use a light source or a laser beam with an appropriate emission band to excite a fluorophore-labeled lipoprotein sample. As the fluorophore-labeled lipoprotein components of the biological sample pass through the detection window of the system, the fluorophore is excited by a laser beam of the appropriate wavelength to induce a signal (i.e., a characteristic fluorescent emission maximum).

In one embodiment, the CE-ITP-LIF and/or MPCE-ITP-LIF systems use one laser beam with an appropriate emission band to excite one fluorophore or non-specific dye. Alternatively, the CE-ITP-LIF and/or MPCE-ITP-LIF systems may use one laser beam with an appropriate emission band to excite one or more fluorophores. Likewise, the CE-ITP-LIF and/or MPCE-ITP-LIF system may use more than one laser beam with the appropriate emission bands to excite two, three, four, five, six, seven, eight, nine, or any number of fluorophores.

In some embodiments, the biological sample and/or signal-producing lipoprotein standard is labeled with a fluorophore-labeled antibody or fluorophore-labeled antibody fragment having a fluorescent emission spectrum that does not significantly overlap with the emission spectrum of the non-specific lipophilic dye. An exemplary antibody fragment is a fragment antigen-binding fragment. Suitable fluorophores are described above are known in the art and may be chosen from a group including, but not limited to, Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, Cy®3, Cy®5, Fluorescein (FITC), Oregon Green®, Pacific Blue™, Pacific Green™, Pacific Orange™, Tetramethylrhodamine (TRITC), Texas Red®, and Texas Red®. In accordance with this embodiment, the CE-ITP-LIF and/or MPCE-ITP-LIF systems comprise one or more lasers with an appropriate emission band to excite the fluorophore-labeled antibody and non-specific lipid dye.

Fluorophore-labeled antibodies may be directed to an apolipoprotein class or subclass-specific epitope. Apolipoproteins are structural components of lipoprotein particles and are bound to water-insoluble lipid molecules by covalent or non-covalent forces in a specific stoichiometry (see U.S. patent application Ser. No. 14/194,142). Apolipoprotein species include, but are not limited to, apolipoprotein A (apoA), apolipoprotein B (apoB), apolipoprotein C (apoC), apolipoprotein D (apoD), apolipoprotein E (apoE), apolipoprotein H (apoH), and apolipoprotein (a). U.S. patent application Ser. No. 14/194,142 describes the association of apolipoprotein particles with specific lipoproteins. Apolipoprotein subclasses include apoA-I, apoA-II, and apoA-IV.

In each of the preceding embodiments, the CE-ITP-LIF and/or MPCE-ITP-LIF system may be equipped with a detector to enable detection of the signal produced by the non-specific lipophilic dye and/or fluorophore-labeled antibody.

In one embodiment, the detector is a multipixel detector. An exemplary multipixel detector is a photodiode array.

Systems with CE-ITP capability are known in the art. An exemplary CE-ITP system is made by deltaDOT Ltd. Such instruments can be modified with one or more modifications to perform the methods of the present invention. For example, in order to use an Alexa Fluor® 488 fluorophore, which is preferentially excited at 488 nm wavelength, in the method of the present invention, a CE-ITP system may be modified comprise a laser with a specific wavelength (e.g., 445 nm, 473 nm, or 488 nm) to illuminate the capillary for the measurement of fluorophore levels migrating past the observation window. Additionally, the system may be modified to comprise a series of optical components (e.g., lenses and filters) in front of the detector (e.g, a photodiode array), to focus the light beam and narrow the wavelength absorbed to that expected from the Alexa-488 fluorophore. Exemplary optical systems of the present invention are described in FIGS. 1-4 and Example 1.

In accordance with this embodiment of the invention, the labelled-lipoprotein is excited by a light source and emits a signal which is detected by a photodiode array, which detects signals over time and space. A computer in communication with the instrument collects the signal emission data and converts it to a form interpretable by a person, such as an electropherogram generated by signal processing algorithms, or a form for further computational analysis.

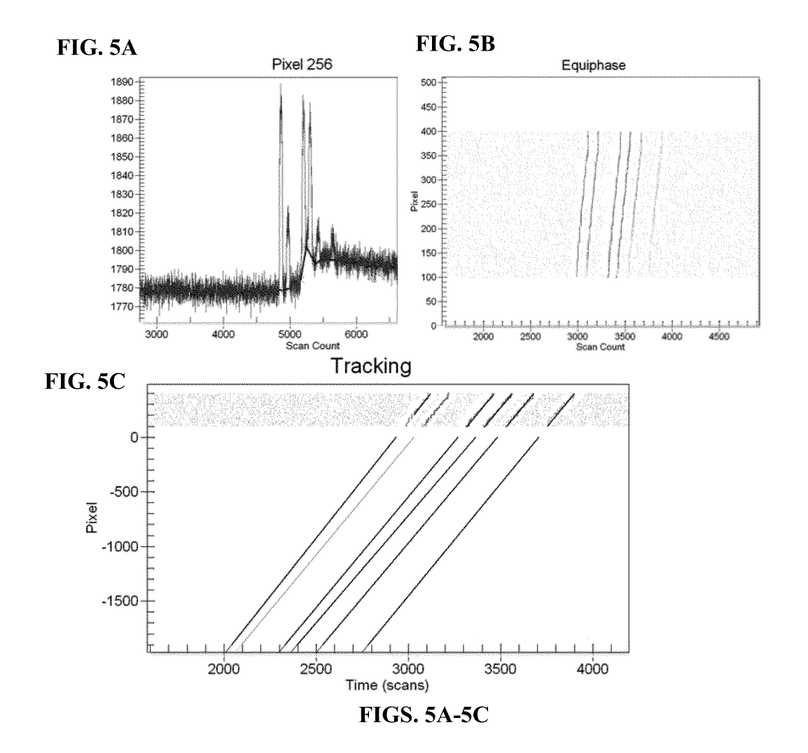

As described herein, an electropherogram is a plot of results recording the separated components of a biological sample produced by capillary electrophoresis (see FIG. 5 and Examples 2-5). The electropherogram may comprise several peaks, each corresponding to the relative molar concentration and/or particle number of a fluorophore-labeled lipoprotein component in the biological sample (see Examples 2-5). The total area under each peak corresponds to the total signal detected in a sample.

To directly measure the phospholipid content of lipoprotein and/or lipid particles in a biological sample, the signal produced by the dye-labeled lipoprotein and/or lipid particles of a biological sample are detected and compared.

The signal produced by a biological sample labeled with a non-specific lipophilic dye is consistent from particle to particle when carried out to saturation. The ratio of the signal produced per unit of phospholipid particle concentration is known as the saturation ratio and is equal to the

$$\frac{signal}{[ phospholipid ]}$$

ratio, where [phospholipid] denotes phospholipid concentration. As described herein, signal-producing lipoprotein standards comprise a known

$$\frac{signal}{[ phospholipid ]}$$

ratio. Determining the

$$\frac{signal}{[ phospholipid ]}$$

ratio of a signal-producing lipoprotein standard involves: (i) providing a known lipid particle composition; (ii) saturating a known lipid particle composition with a non-specific lipophilic dye; (iii) analyzing the saturated known lipid particle composition using a CE-ITP-LIF system, wherein the analyzing involves detecting the fluorescent output of the saturated known lipid particle composition; (iv) comparing the fluorescent output of the saturated known lipid particle composition to the known concentration of the saturated lipid particle composition; and (v) generating a

$$\frac{signal}{[ phospholipid ]}$$

constant ratio.

In accordance with the methods of the present invention, the

$$\frac{signal}{[ phospholipid ]}$$

ratio may be determined prior to subjecting the dye-labeled lipoprotein to a CE-ITP-LIF system. For example, the ratio of lipophilic dyes to phospholipid may be determined experimentally prior to an analysis of the biological sample. The

$$\frac{signal}{[ phospholipid ]}$$

concentration ratio may alternatively be determined through a series of experiments on a variety of particles. Accordingly, particles may be first purified into particle classes. Each class, saturated with lipophilic dyes, may be separately analyzed. Additionally, each class may be characterized by its subclass component for

$$\frac{signal}{[ phospholipid ]}$$

ratio. Accordingly, a standard measurement of the saturation ratio is produced to calculate the concentration of each particle from a spectrum.

By way of example, a biological sample labeled to saturation with NBD ceramide would produce the same saturation ratio

$$( i . e . , \frac{NBD signal}{[ phospholipid ]} )$$

for each of the HDL, LDL, VLDL, IDL, and Lp(a) particles in the biological sample.

Detecting and comparing signals produced by the non-specific lipophilic dye and the signal-producing lipoprotein standard allows for the determination of the phospholipid concentration of a lipoprotein and/or lipid particle class or subclass in a biological sample, which is required for quantifying the molar concentration and/or particle number of a lipoprotein and/or lipid particle present in a biological sample. Quantifying the molar concentration and/or particle number of a lipoprotein and/or lipid particle present in a biological sample also requires knowledge of (i) the relationship between the surface area of a phospholipid head group to the surface area of a particular spherical lipoprotein and (ii) the percentage of phospholipids in surface area of the lipoprotein

As described herein, analysis of the correlations between size and chemical composition of lipoproteins of normolipidemic human plasma shows that the structure of all circulating lipoproteins is consistent with a spherical model of radius ‘r’ in which a core region is surrounded by an outer surface of lipid particles (see Shen et al., “Structure of Human Serum Lipoproteins Inferred from Compositional Analysis,” Proc. Natl. Acad. Sci. USA. 74(3): 837-841 (1977), which is hereby incorporated by reference in its entirety). The hydrophilic head group of phospholipids at the outer surface of the lipoprotein particle has a surface area equal to 62.7 Å²/molecule (see Shen et al., “Structure of Human Serum Lipoproteins Inferred from Compositional Analysis,” Proc. Natl. Acad. Sci. USA. 74(3): 837-841 (1977), which is hereby incorporated by reference in its entirety). The surface area of a spherical lipoprotein or lipid particle is equal to 4πr², where r is equal to the radius in Angstroms of a lipoprotein or lipid particle. Thus, the number of phospholipid (PL) particles comprising a single lipoprotein or lipid particle can be determined based on the spherical nature of a lipoprotein particle and its relationship to the physical properties of phospholipids, as shown in the following formula:

$$\frac{\# PL particles}{lipoprotein particle} = \frac{\% PL ( 4 π {r}^{2} )}{62 {Å}^{2} PL} ;$$

where % PL is the percent phospholipid in the surface area of the lipoprotein.

When the phospholipid concentration of a sample is known (e.g., in mg/dL), the concentration of lipid molecules (e.g., in molecules/L) in the sample can be determined based on the following calculations:

$$\frac{\# PL molecules}{L} = [ PL ] \times \frac{10 dL}{1 L} \times \frac{1 g}{1 , 000 mg} \times \frac{mol}{775 g} \times \frac{6.02 \times {10}^{23} molecules}{1 mol} ;$$

where [PL] is the concentration of phospholipid in mg/dL and 775 g is equivalent to the molecular weight of 1 mol of PL.

Likewise, the number of lipoprotein particles in a sample fraction can be determined using the formula:

$$\frac{\# lipoprotein particles}{L} = \frac{( \frac{\# PL molecules}{L} )}{( \frac{\# PL particles}{lipoprotein particle} )}$$

Moreover, the lipoprotein or lipid particle number (“PN”; concentration of lipoprotein particles in

$$\frac{n mol}{L} )$$

of a sample fraction can be determined using the formula:

$$PN = \frac{( \frac{\# PL molecules}{L} )}{( \frac{\# PL particles}{lipoprotein particle} )} \times \frac{1 mol}{6.02 \times {10}^{23} molecules} \times \frac{{10}^{9} nmol}{1 mol} ,$$

which is equivalent to:

$$\begin{matrix}PN = [ PL ] \times \frac{( \frac{( 10 dL ) ( 6.02 \times {10}^{23} molecules )}{( L ) ( 1 , 000 mg ) ( 775 )} )}{( \frac{\% PL ( 4 π {r}^{2} )}{62 {Å}^{2} PL} )} \times \\ \frac{( {10}^{9} nmol )}{( 6.02 \times {10}^{23} molecules )} \\ = [ PL ] \times \frac{( 10 dL ) ( 6.02 \times {10}^{23} molecules ) ( 62 {Å}^{2} ) ( {10}^{9} nmol )}{( L ) ( 1 , 000 mg ) ( 775 ) ( \% PL ) ( 4 π {r}^{2} ) ( 6.02 \times {10}^{23} molecules )} II \\ = [ PL ] \times \frac{( 10 dL ) ( 62 {Å}^{2} ) ( {10}^{9} nmol )}{( L ) ( 1 , 000 mg ) ( 775 ) ( \% PL ) ( 4 π {r}^{2} )} III \\ = [ PL ] \times \frac{63662 {Å}^{2} nmol dL}{( L ) ( mg ) ( \% PL surface area ) ( {r}^{2} )} ; IV\end{matrix} \qquad I$$

where PN is the particle number of the lipoprotein in

$$( \frac{n mol}{L} ) ;$$

[PL] is phospholipid concentration of the lipoprotein in

$$( \frac{mg}{dL} ) ;$$

r² is the radius of the lipoprotein in (Å) squared; and % PL is the percent phospholipid in the surface area of the lipoprotein.

As an example of the quantifying step of the methods described herein, the outer surface area of an LDL particle, with amino acid corrections, was calculated to be 38.1% as follows:

$$\begin{matrix}\frac{1830 amio acids}{LDL particle} \times \frac{15.6 {Å}^{2}}{amino acid molecules} = \frac{75 , 348 {Å}^{2}}{LDL particle} & I \\ \frac{653 PL molecules}{LDL particle} \times \frac{17 {Å}^{2}}{PL particle} = \frac{46 , 363 {Å}^{2}}{LDL particle} & II \\ Total LDL particle surface area = \frac{75 , 348 {Å}^{2}}{LDL particle} + \frac{46 , 363 Å}{LDL particle} \\ = \frac{121 , 711 {Å}^{2}}{LDL particle} & \begin{matrix}\begin{matrix}III \\ \end{matrix} \\ IV\end{matrix} \\ \% PL surface area = \frac{\frac{46 , 363 {Å}^{2}}{LDL particle}}{\frac{121 , 711 {Å}^{2}}{LDL particle}} = 38.1 \% & V\end{matrix}$$

Accordingly, the PN number for an LDL sample with a PL concentration of 150 mg PL/dL, a 38.1% PL surface area, and a radius equal to 96 Å would be calculated as follows:

$$\begin{matrix}PN = [ PL ] \times \frac{63662 {Å}^{2} nmol dL}{( L ) ( mg ) ( \% PL ) ( {r}^{2} )} & I \\ PN = ( \frac{150 mg}{dL} ) \times \frac{63662 {Å}^{2} nmol dL}{( L ) ( mg ) ( 38.1 \% ) ( {( 96 Å )}^{2} )} = 2 , 720 nmol \text{/} L ; & II\end{matrix}$$

where PN is the particle number of the lipoprotein in

$$( \frac{n mol}{L} ) ;$$

[PL] is phospholipid concentration of the lipoprotein in

$$( \frac{mg}{dL} ) ;$$

r² is the radius of the lipoprotein in (Å) squared; and % PL is the percent phospholipid in the surface area of the lipoprotein.

In each of the preceding embodiments of the invention, the signal produced by the signal-producing lipoprotein standard is measured and compared with the signal produced from the lipophilic dye-labeled lipoprotein or lipid particle and the molar concentration and/or particle number of the lipoprotein or lipid particle is determined based on the following formula:

$$PN = [ PL ] \times \frac{63662 {Å}^{2} nmol dL}{( L ) ( mg ) ( \% PL ) ( {r}^{2} )} ;$$

where PN is the particle number of the lipoprotein in

$$( \frac{nmol}{L} ) ;$$

[PL] is phospholipid concentration of the lipoprotein in

$$( \frac{mg}{dL} ) ;$$

r² is the radius of the lipoprotein in (Å) squared; and % PL is the percent phospholipid in the surface area of the lipoprotein.

In accordance with this aspect of the invention, the lipoprotein and/or lipid particle [PL], r², and % PL values are proportional to the signal-producing standard [PL], r², and % PL values.

A second aspect of the invention relates to a method of assessing a health risk in an individual. This method involves determining the particle number and/or molar concentration of a lipoprotein or lipid particle in a biological sample from a subject according to the first aspect of the invention. The method further involves assessing the health risk of the subject based on the particle number and/or molar concentration of the lipoprotein or lipid particle.

In one embodiment, the health risk is associated with a cardiovascular disorder, a metabolic disorder, or diabetes.

It is well-established that the lipoprotein subclass distribution profile of an individual may be indicative of a health risk. In particular, cardiovascular and metabolic disorders are correlated strongly with specific patterns of subclass quantity and size (see U.S. Pat. No. 6,518,069).

Various disease states, including but not limited to cardiovascular disease, liver disease, and diabetes mellitus, are associated with the levels of apolipoproteins and/or lipoprotein particles (see, e.g., U.S. Pat. No. 6,518,064). For example, apoB is a constituent of VLDL and LDL particles, which are associated with increased risk of cardiovascular disease. Increased levels of Lp(a), which comprise an LDL-like particle with apoA bound to apoB by a disulfide bond, is associated with an increased risk of early atherosclerosis independent of other cardiac risk factors. Moreover, differences in the amount of cholesterol in a particle may also correlate with the risk of cardiovascular disease. For example, elevated levels of small, dense, cholesterol ester rich LDL correlate with an increased risk of cardiovascular disease; while elevated levels of cholesterol rich HDL correlate with a decreased in risk of cardiovascular disease. Thus, the risk of developing a cardiovascular disease can be assessed by quantifying the levels of these lipoproteins.

In one embodiment, the subject is a mammal selected from the group including, but not limited to, a human, a non-human primate, a rodent, a canine, a feline, and a bovied.

In another embodiment, the subject is a human.

The subject may be healthy. Alternatively, the subject may be known to suffer from a cardiovascular or metabolic disorder and/or at risk of suffering from a cardiovascular or metabolic disorder. The subject may be a patient suspected of suffering from a lipoprotein-associated disorder including, but not limited to, cardiovascular disorders and obesity. Additional lipoprotein disorders include hyperlipidemia (i.e., the abnormal elevation of lipids or lipoproteins in the blood), arteriovascular disease, atherosclerosis, pancreatitis, and liver disorders. Moreover, elevated or unbalanced lipid and lipoprotein levels are reflective of a subject's development of or progression of diabetic conditions and metabolic disorders.

As described above, suitable biological samples according to the invention include, without limitation, fresh blood, stored blood, or blood fractions.

The method involves (a) contacting a biological sample with a non-specific lipophilic dye under conditions suitable for the non-specific lipophilic dye to bind to the lipoprotein, or a lipid particle thereof, to form a lipophilic dye-labeled lipoprotein, wher the biological sample comprises a signal-producing lipoprotein standard; (b) subjecting the dye-labeled lipoprotein to a capillary isotachophoresis laser-induced fluorescence (CE-ITP-LIF) system; (c) detecting and comparing signals produced by the non-specific lipophilic dye and the signal-producing lipoprotein standard; and (d) quantifying, based on said detecting and comparing, the molar concentration and/or particle number of the lipoprotein or lipid particle in the sample, wherein the detected signals are proportional to the molar concentration and/or particle number of the lipoprotein or lipid particle in the sample.

In some embodiments, the CE-ITP-LIF system separates the components of the sample from one another along a common capillary. In other embodiments, the CE-ITP-LIF system is a multiplex capillary isotachophoresis laser induced fluorescence (MPCE-ITP-LIF) system. In accordance with these embodiments, the MPCE-ITP-LIF system separates multiple samples simultaneously.

In each of the preceding embodiments, the CE-ITP-LIF and/or MPCE-ITP-LIF system may be equipped with an appropriate detection device to enable detection of the signal produced by the fluorophore-labeled lipoprotein and/or signal-producing calibrator lipoprotein.

In one embodiment, the detector is a multipixel detector. An exemplary multipixel detector is a photodiode array.

In one embodiment, the signal-producing lipoprotein standard comprises a standard lipoprotein or lipid particle with a known concentration, a known radius, a known lipid concentration, a known lipid distribution, or a combination thereof.

In accordance with this embodiment, the signal produced by the signal-producing lipoprotein standard is measured and compared with the signal produced from the lipophilic dye-labeled lipoprotein or lipid particle and the molar concentration and/or particle number of the lipoprotein or lipid particle is determined based on the following formula:

$$PN = [ PL ] \times \frac{63662 {Å}^{2} nmol dL}{( L ) ( mg ) ( \% PL ) ( {r}^{2} )} ;$$

where PN is the particle number of the lipoprotein in

$$( \frac{nmol}{L} ) ;$$

[PL] is phospholipid concentration i of the lipoprotein in

$$( \frac{mg}{dL} ) ;$$

r² is the radius of the lipoprotein in (Å) squared; and % PL is the percent phospholipid in the surface area of the lipoprotein. The lipoprotein and/or lipid particle [PL], r², and % PL values may be proportional to the signal-producing standard [PL], r², and % PL values.

This aspect of the invention involves assessing the cardiovascular risk of the subject based on the particle number and/or molar concentration of the lipoprotein in a biological sample from a subject.

Lipoprotein particle profiles are different for different individuals and for the same individual at different times. The lipoprotein particles or portions thereof to be assessed for determining a health risk include, but are not limited to, VLDL, LDL, IDL, HDL, chylomicron, lipoprotein X, Lp(a), and subforms and mixtures thereof.

Chylomicrons are produced in the intestine and transport digested fat to the tissues. Lipoprotein lipase hydrolyzes triacylgylcerol to form fatty acids. Chylomicrons are one of the largest buoyant particles. VLDL is formed from free fatty acids upon metabolism of chylomicrons in the liver. Lipoprotein lipase hydrolyzes triacylgylcerol to form fatty acids. IDL is the unhydrolyzed triacylglycerol of VLDL. IDL becomes LDL due to hepatic lipase. HDL plays a role in the transfer of cholesterol to the liver from peripheral tissue. HDL is synthesized in the liver and intestines.

LDL particles bind to LDL receptors. Upon receptor binding, LDL is removed from the blood. Cells use cholesterol within the LDL for membranes and hormone synthesis. LDL deposits LDL cholesterol on the arterial wall which contributes to cardiovascular disease. LDL causes inflammation when it builds up inside an artery wall. Macrophages are attracted to the inflammation and tum into foam cells when they take up LDL, causing further inflammation. Smaller, denser LDL contain more cholesterol ester than the larger, buoyant LDL.

The structure of the LP(a) is that of an LDL-like particle with apolipoprotein A bound to apolipoprotein B by a disulfide bond. Lp(a) particles appear to play a role in coagulation and may stimulate immune cells to deposit cholesterol on arterial walls. A high Lp(a) level indicates a higher risk for cardiovascular disease. Therefore, Lp(a) is useful in diagnostic and statistical risk assessment. Lp(a) may serve to facilitate LDL plaque deposition. Levels of Lp(a) are increased in atherogenic events.

Lp(a) may have a link between thrombosis and atherosclerosis, interfering with plasminogen function in the fibrinolytic cascade. Numerous studies have documented the relationship of high plasma Lp(a) concentrations to a variety of cardiovascular disorders, including peripheral vascular disease, cerebrovascular disease, and premature coronary disease. One large study of older Americans, in particular, demonstrated elevated levels of Lp(a) independently predict an increased risk of stroke, death from vascular disease, and death from all causes in men (see Fried et al., “The Cardiovascular Health Study: Design and Rationale,” Ann. Epidemiol. 3:263-76 (1991), which is hereby incorporated by reference in its entirety).

In one embodiment of the methods of the present invention, the particle number and/or molar concentration of a lipoprotein or lipid particle in a biological sample is used to determine the lipoprotein distribution of the biological sample. The lipoprotein distribution may comprise the relative amounts of each lipoprotein and/or lipid particle in a biological sample. The lipoprotein distribution may also state the particle number and/or molar concentration of a lipoprotein or lipid particle in a biological sample.

In another embodiment, the subject is assigned to one of a low, moderate, or high health risk categories based on the particle number and/or molar concentration of the lipoprotein. In other embodiments, the health risk is a risk associated with a cardiovascular disorder, a metabolic disorder, or diabetes.

There are well established recommendations for cut-off values for biochemical markers (for example, and without limitation, lipoprotein levels) for determining a health risk. For instance, the cut-off values for assigning such risk categories may be as follows: Lp(a): <75 nmol/L optimal, 76-125 nmol/L intermediate risk, >126 nmol/L high risk; LDL: <1000 nmol/L optimal, 1000-1299 nmol/L intermediate risk, >1300 nmol/L high risk.

In some embodiments, the method further comprises administering to the subject a therapeutic regimen for reducing the health risk, or modifying an existing therapeutic regimen for the subject for reducing the health risk, based on the health risk category assigned to the subject. In accordance with this embodiment of the present invention, the therapeutic regimen comprises administering a drug and/or a supplement or the existing therapeutic regimen comprises administering a modified dose of a drug and/or a supplement. The drug or supplement may be any suitable drug or supplement useful for the treatment or prevention of diabetes and related cardiovascular disease.

In some embodiments, the drug is selected from the group consisting of niacin, an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, an angiotensin system inhibitor, and combinations thereof. Likewise, the drug may be selected from the group consisting of niacin, statin, ezetimibe, fenofibrate, estrogen, raloxifene and combinations thereof.

The agent is administered in an amount effective to treat the cardiovascular disorder, metabolic disorder, diabetes, or any combination thereof or to lower the risk of the subject for developing a future cardiovascular disorder, metabolic disorder, diabetes, or any combination thereof.

In some embodiments, the selected therapeutic regimen involves giving recommendations on making or maintaining lifestyle choices based on the results of said health risk determination. In accordance with this embodiment, the lifestyle choices involve changes in diet, changes in exercise, reducing or eliminating smoking, or a combination thereof.

In any of the preceding embodiments according to this aspect of the invention, the biological sample is selected from the group consisting of blood, plasma, urine and saliva.

In any of the preceding embodiments according to this aspect of the invention, the non-specific lipophilic dye is selected from the group consisting of NDB-ceramide, ADIFAB fatty acid indicators, phospholipids with BODIPY dye-labeled acyl chains, phospholipid with DPH-labeled acyl chains, phospholipids with NBD-labeled acyl chains, phospholipids with pyrene-labeled acyl chains, phospholipids with a fluorescent or biotinylated head groups, LipidTOX phospholipid, neutral lipid stains and combinations thereof.

A third aspect of the invention relates to a system for determining the molar concentration and/or particle number of a spherical lipoprotein or lipid particle in a biological sample. This system comprises a capillary electrophoresis apparatus for separating components of a moiety-bound sample, wherein the moiety-bound sample is prepared by contacting the biological sample with a fluorophore-labeled antibody under conditions suitable for the fluorophore-labeled antibody to bind to the lipoprotein or an immunologically active component thereof, to form a fluorophore-labeled lipoprotein. The system also comprises a detector for detecting signals produced by the fluorophore-labeled lipoprotein and a processor for quantifying, based on said detecting, the concentration and/or particle number of the lipoprotein in the sample, where the detected signals are proportional to the molar concentration and/or particle number of the lipoprotein in the sample.

The system comprises a separation apparatus to isolate lipoprotein particles and lipoprotein subclasses in the bodily fluid based on their ionic mobilities using a capillary electrophoresis (CE-ITP) apparatus and a detector for detecting signals indicating the presence of labeled-lipoprotein particles. In one embodiment, the system is a capillary isotachophoresis laser-induced fluorescence (CE-ITP-LIF) system. In accordance with this embodiment, the system is a multiplex capillary isotachophoresis laser induced fluorescence (MPCE-ITP-LIF) system. MPCE-ITP-LIF systems are described in detail above, in FIGS. 1-4, and Example 1 of the present application. In accordance with this embodiment, the MPCE-ITP-LIF system separates multiple samples simultaneously.

The CE-ITP and or MCPE-ITP system further comprises a laser-induced fluorescence (LIF) detector for detecting a signal emitted from the fluorescent dye or a fluorophore label. The signal is used to quantitate the level of said specific lipoprotein particles.

In one embodiment, the system is a CE-ITP-LIF system. In another embodiment, the system is an MCPE-LIF system.

As noted in the accompanying Figures (FIGS. 1-4) and Example 1, the apparatus may include a laser and set of optical components such as lenses and filters. Lasers may be used to excite the labelled lipoprotein particle. Filters may be used to limit light hitting the detectors by intensity, focal length and wavelength, so that only the fluorophore of interest is monitored.

In an example, an Alexa Flour® 488 fluorophore may be used to label and detect a specific lipoprotein or lipid particle. Alternatively, carboxyfluorescein may be used to detect the phospholipids of a lipoprotein particle. In order to measure the signal produced by an Alexa Flour® 488 labeled lipoprotein or lipid particle, the system may be equipped with a 488 nm laser. Alternatively, the system may be equipped with a 408 nm laser in order to detect a carboxyfluorescein labeled lipoprotein particle.

In one embodiment, the separation apparatus is flanked by two optical zones. The first optical zone may comprise a specific wavelength laser. The second optical zone may comprise a detector to execute laser-induced fluorescence measurements.

The detector detects the signal produced by a labeled apolipoprotein or lipoprotein particles. In one embodiment, the detector is a multipixel detector. An exemplary multipixel detector is a photodiode array.

The system also comprises a processor connected to the detector to process the detected fluorescent signal into an output value for interpretation by another processor or a human. In one embodiment, the processor is programmed with signal processing algorithms to process the signal by (i) reading the signal in a time dependent manner from a selected pixels on a multipixel detector such as a photo diode array; (ii) interpreting the read signal with those algorithms to filter noise, compute wavelength or frequency value from the input signal, perform a quality assessment of the computed values; and (iii) producing an output value for further analysis. The further analysis may comprise human interpretation or additional computational processing.

In many cases, the output is an electropherogram showing the detected signals as peaks for identification and analysis. As described above, an electropherogram is a plot of results recording the separated components of a biological sample produced by capillary electrophoresis (see FIG. 5 and Examples 2-5). The electropherogram may comprise several peaks, each corresponding to the relative molar concentration and/or particle number of a fluorophore-labeled lipoprotein component in the biological sample (see Examples 2-5). The total area under each peak corresponds to the total signal detected in a sample.

Some dimension or representation of a signal's peak may be proportional to the molar concentration of the apolipoprotein or lipoprotein particle of interest. The output value may also be indicative of the risk of developing a cardiovascular or metabolic disorder.

Signal processing may be accomplished using various technologies known in the art. An exemplary technology for use in signal processing according to the present invention is deltaDOT's multipixel detection technology. A unique property of deltaDOT's multipixel detection technology is that it allows the tracking of each analyte peak as it moves across the capillary viewing region. By taking multiple images of the analyte at different spatial positions a direct measurement of the velocity of each peak as it traverses the 512 pixel photo diode array may be obtained.

The tracking concept and general principle is illustrated in FIGS. 5A-5C. The analysis consists of three stages. First, peak searching is performed on each individual pixels electropherogram. Each peak detected is quantified in terms of migration time and peak area (or peak height). Next the algorithm sorts through all of the peaks and tries to assign them to tracks, which represents the path of the analytes across the capillary window. Once a set of peaks has been assigned to a track, a linear fit is used to determine the velocity of the analyte averaged across all of the pixels.

The system may further comprise a storage module for the output value thus obtained. Further, the system comprises a module for generating a report based on output value for the user.

The report may include, among other things, the molar concentration and/or particle number of a fluorophore-labeled apolipoprotein and/or lipoprotein in a biological sample; an output value indicative of the risk of developing a cardiovascular disease or metabolic disorder; and a description of a recommended treatment regimen based on a cardiovascular disease or metabolic disorder risk assessment.

In some embodiments, the results of lipoprotein analyses are reported in such a report. A report refers in the context of lipoprotein and other lipid analyses to a report provided, for example to a patient, a clinician, other health care provider, epidemiologist, and the like, which includes the results of analysis of a biological specimen, for example a plasma specimen, from an individual. Reports can be presented in printed or electronic form, or in any form convenient for analysis, review and/or archiving of the data therein, as known in the art.

A report may include identifying information about the individual subject of the report, including without limitation name, address, gender, identification information (e.g., social security number, insurance numbers), and the like.

A report may include biochemical characterization of the lipids in the sample in addition to Lp(a), for example without limitation triglycerides, total cholesterol, LDL cholesterol, and/or HDL cholesterol, and the like.

The term “reference range” and like terms refer to concentrations of components of biological samples known in the art to reflect typical normal observed ranges in a population of individuals. A report may further include characterization of lipoproteins, and reference ranges therefore, conducted on samples prepared by the methods provided herein.

Exemplary characterization of lipoproteins in an analysis report may include the concentration and reference range for VLDL, IDL, Lp(a), LDL and HDL, and subclasses thereof. A report may further include lipoprotein size distribution trends.

### EXAMPLES

The following examples are provided to illustrate embodiments of the present invention but they are by no means intended to limit its scope.

### Example 1

### Optical Apparatus for Use in CE-ITP-LIF Systems

A schematic of an optical apparatus comprising two optical zones for use in a CE-ITP-LIF system is shown in FIG. 1. Optics zone 1 comprises an optical rail on which are arranged a 445 nm or other specific wavelength laser or laser diode. Light from these sources is focused through a series of optical components comprising, but not limited to, a line generator, a crossed linear polarizer, and a neutral density filter. Light from optics zone 1 is focused onto a 12.5 mm area of a 100 μM internal diameter fused silica capillary (˜365 μM o.d.) in which a 20 mm viewing window has been created by thermal removal of the polyamide sheath. The light then passes through the sample that is being separated by ITP and excites the fluorescent label attached to each analyte molecule (e.g., a lipoprotein and/or lipid particle). Emitted light energy, at a wavelength specific to the fluorescent label is then focused onto a 512 pixel photo diode array (“PDA”) through another series of optical components in optics zone 2. Optics zone 2 comprises a set of imaging lenses (e.g., convex lenses), and an orthogonal crossed linear polarizer. After passing through a cut-on filter that transmits above a certain wavelength, the light energy reaches the detector where the data is acquired on the PDA and the signal is processed by signal processing algorithms.

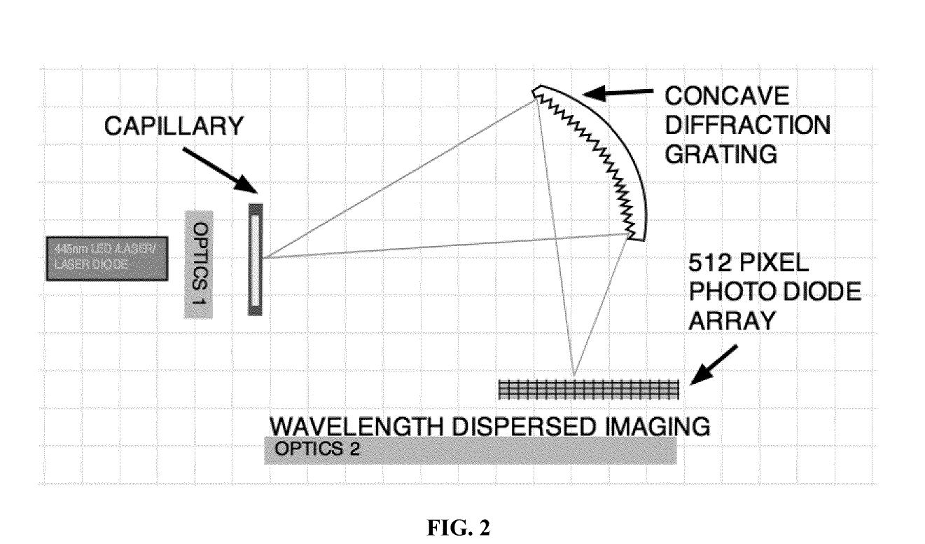

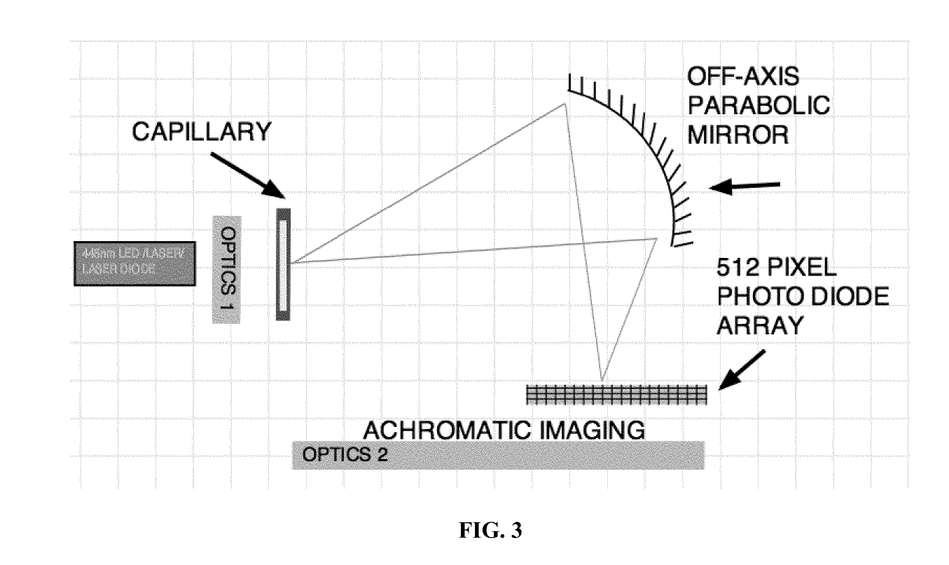

FIG. 2 shows an optical apparatus with a 445 nm LED/Laser/Laser Diode in optics zone 1 and an off axis concave diffusion grating in optics zone 2. The diffusion grating focusses wavelength dispersed achromatic light of a wavelength specific to the fluorescent label onto the 512 pixel photo diode array. By rotating the diffusion grating, the light energy reaches the detector where the data is acquired on the PDA and the signal is processed by proprietary signal processing algorithms. An additional cut-on filter or crossed polarizer may be added. A simple off axis parabolic mirror may replace the diffusion grating (FIG. 3).

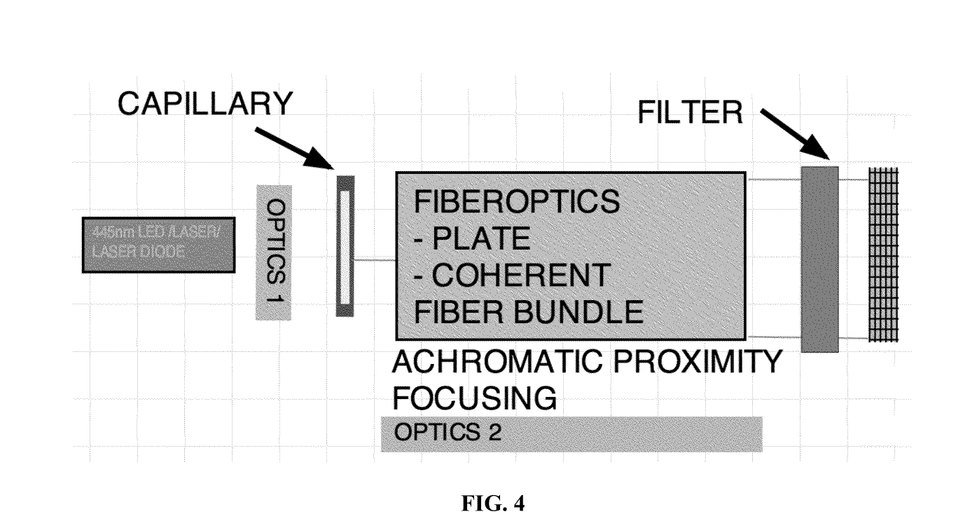

FIG. 4 is a schematic of an optical system comprising a fibre-optic plate (“FOP”) or coherent fibre bundle in optics zone 2. This configuration allows for proximity focusing via a cut-on filter without needing the PDA to touch the capillary (FIG. 4).

### Materials and Methods for Examples 2-5

Leading and terminating electrolytes. The leading electrolyte consists of 10 mm HCL, 0.3% w/v hydroxypropylmethylcellulose (“HPMC”), and 17 mM 2-amino-2-methyl-1,3-propanediol (“Ammediol”). The terminating electrolyte contained 20 mM alanine, 17 mM Ammediol, and was adjusted to pH 10.6 with saturated barium hydroxide solution.

Preparation of Spacer Solutions.

Spacer solutions were prepared to a concentration of 0.32 mg/ml in deionized water and stored at 4° C. Various spacers were made from stock solutions of the following compounds: N-2-acetamido-2-aminoethanesulfonic acid (“ACES”), D-glucuronic acid, octane-sulfonic acid, 2-[(2-Hydroxy-1,1-bis(hydroxymethyl) ethyl)amino]ethanesulfonic acid, 3-[[1,3-dihydroxy-2-(hydroxymethyl)propan-2-yl]amino]propane-1-sulfonic acid, serine, glutamine; methionine, and glycine.

Preparation of the Internal Standard.

1 mg/ml and 2.8 mg.ml carboxyfluorescein (“CF”) solution was prepared in deionized water (“DI”) water and isolated from light.

Biological Samples.

Biological samples were prepared from patients identified as 1, 2, 3, 4, 5, 6, 7, and 8. Patient samples 1, 2, and 6 were previously identified as Lp(a) positive. Patient sample 4 was previously identified as negative for Lp(a).

Biological Sample Preparation.

Biological samples comprising lipoproteins were stained with the fluorescent lipophilic dye 7-nitro-benz-2-oxa-1,3-diazole (“NBD”) ceramide. Briefly, 5 μl of a biological sample were diluted in 37.5 μl deionized water. The diluted sample was incubated for 1 minute with 20 μl NBD-ceramide solution (0.5 mg/ml in ethylene glycol:DMSO, 9:1 (v/v)), mixed with 100 μl of spacer solution (0.32 mg/ml), and spiked with 2.5 μl of the carboxyfluorescein internal standard. In some instances, NBD-ceramide was omitted and replaced with 20 μl of DI water. For biological samples evaluated in the presence of a lipoprotein spike, 2.5 μl of the biological sample was combined with 2.5 μl of the lipoprotein spike prior to dilution in deionized water.

Sample Loading and Data Acquisition.

Samples were injected into a 20 cm Rxi capillary (100 μm) using pressurized injection for 9 seconds at 1 psi. Separation was performed at constant 8 kV. The separated zones were monitored with laser-induced fluorescence detection (excitation 445 nm; emission 550 nm).

Data Analysis and Signal Processing.

Data analysis consists of three stages. First, peak searching is performed on each individual pixel electropherogram (FIG. 5A). Each detected peak is quantified in terms of migration time and peak area (or peak height). Peak area correlates to the particle number of a detected analyte. Next, an algorithm sorts through all of the detected peaks and assigns them to tracks, which represent the path of the analytes across the capillary window (FIG. 5B). Once a set of peaks has been assigned to a track, a linear fit is used to determine the velocity of the analyte averaged across all of the pixels (FIG. 5C), which is needed for signal averaging between pixels.

### Example 2

### Replicate Lipoprotein Profiles of a Single Biological Sample

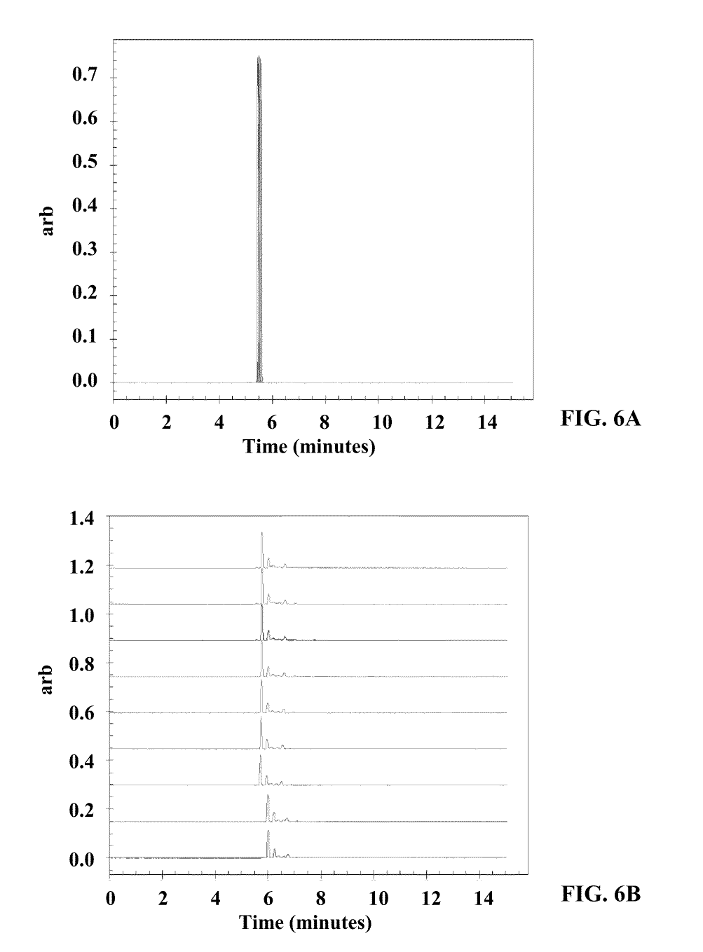

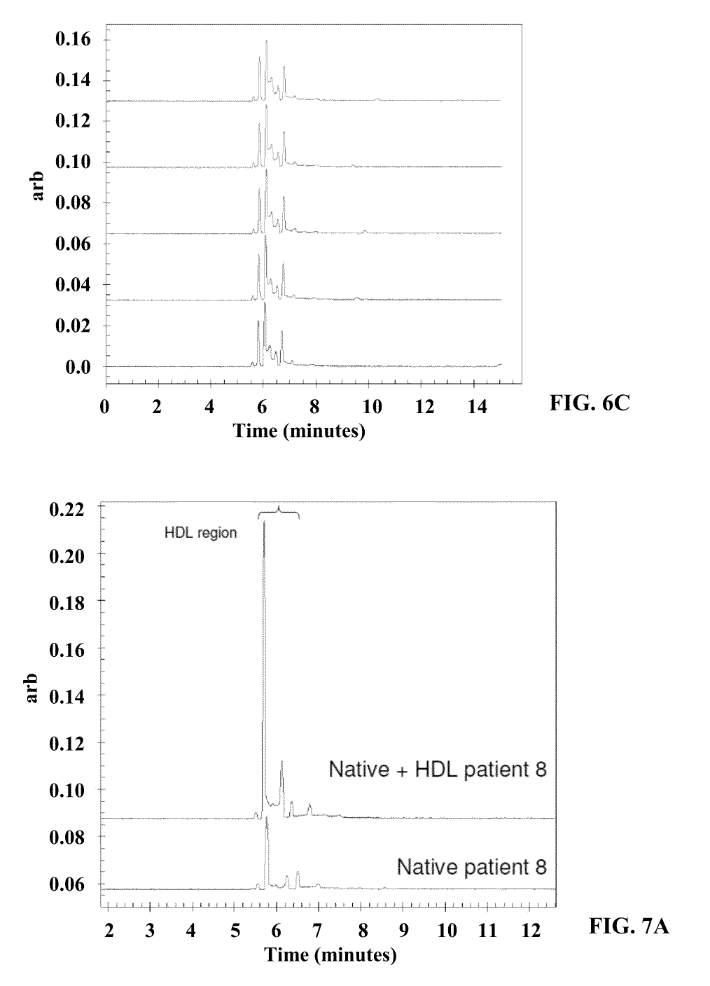

To test the reproducibility of the CE-ITP-LIF system, several replicate biological samples from a single patient were evaluated. As a control experiment, the non-specific lipophilic dye CF was run on the ITP system in the absence of a biological sample. FIG. 6A shows an electropherogram of the control experiment with a peak corresponding to CF (migration time=0.7999), area under peak=2.345). Next, lipoprotein particles in replicate biological samples from patient 8 were labeled with CF and run with a standard CF sample. FIG. 6B is an electropherogram showing the lipoprotein profile of each replicate sample tested. The lipid profile remains constant even after CF has degraded (FIG. 6C).

### Example 3

### Lipoprotein Particle Spiking Results in a Marked Increase in the Corresponding Detected Lipoprotein Peak Height

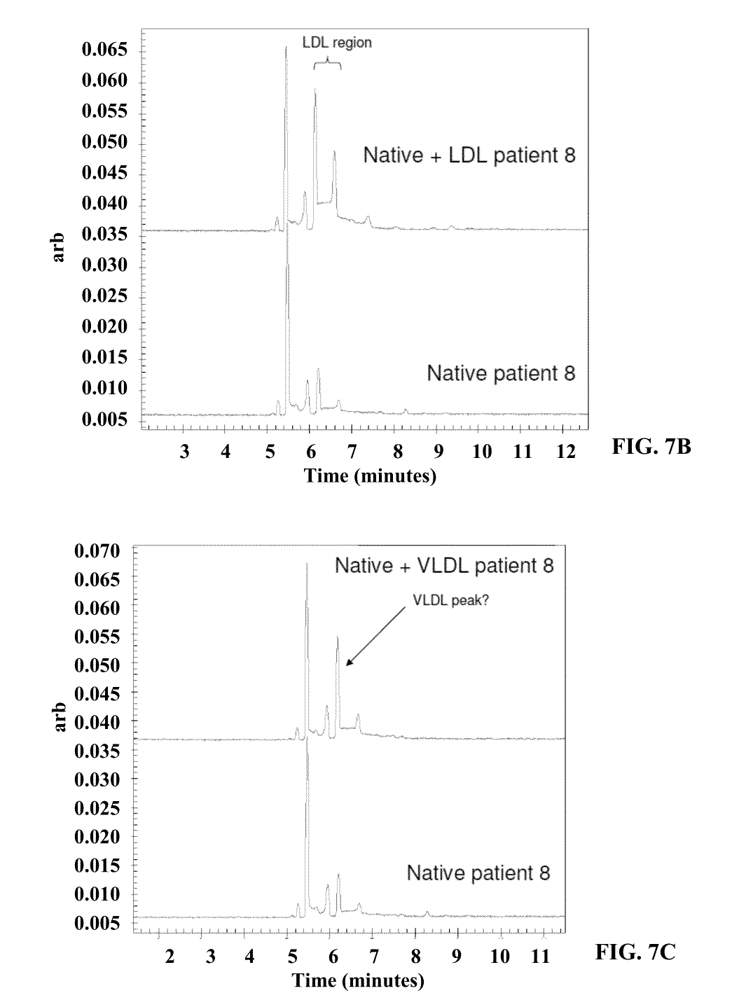

The lipoprotein profile of a biological sample stained with NBD-ceramide generates several peaks corresponding to individual serum lipoproteins (FIGS. 6A-6B). To validate the identity of each individual lipoprotein peak, biological samples were spiked with known amounts of purified lipoprotein. To validate peaks corresponding to HDL and LDL, native samples from patient 8 were spiked with purified HDL and LDL, respectively. The lipid profile of the HDL spiked sample (FIG. 7A, top) and the LDL spiked sample (FIG. 7B, top) were aligned with the lipid profile generated by the native sample (FIG. 7A, bottom; FIG. 7, bottom). As shown in FIG. 7A, there was a marked increase in the peak height and area under the peak in the HDL spiked sample compared to the native sample. FIG. 7B shows the same relationship between the LDL spiked sample compared to the native sample. FIG. 7C shows the lipid profile of a VLDL spiked sample compared to a native sample from patient 8. The VLDL peak (FIG. 7, arrow) seems to fall within the region identified by the LDL spiked sample in FIG. 7B.

### Example 4

### Evaluation of Multiple Biological Samples

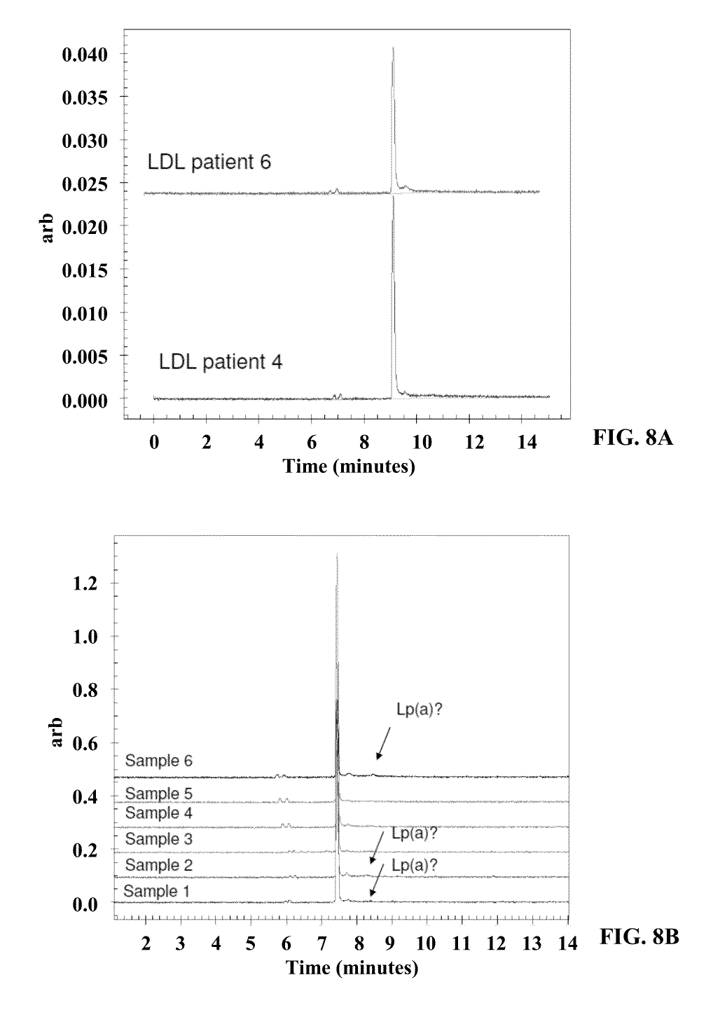

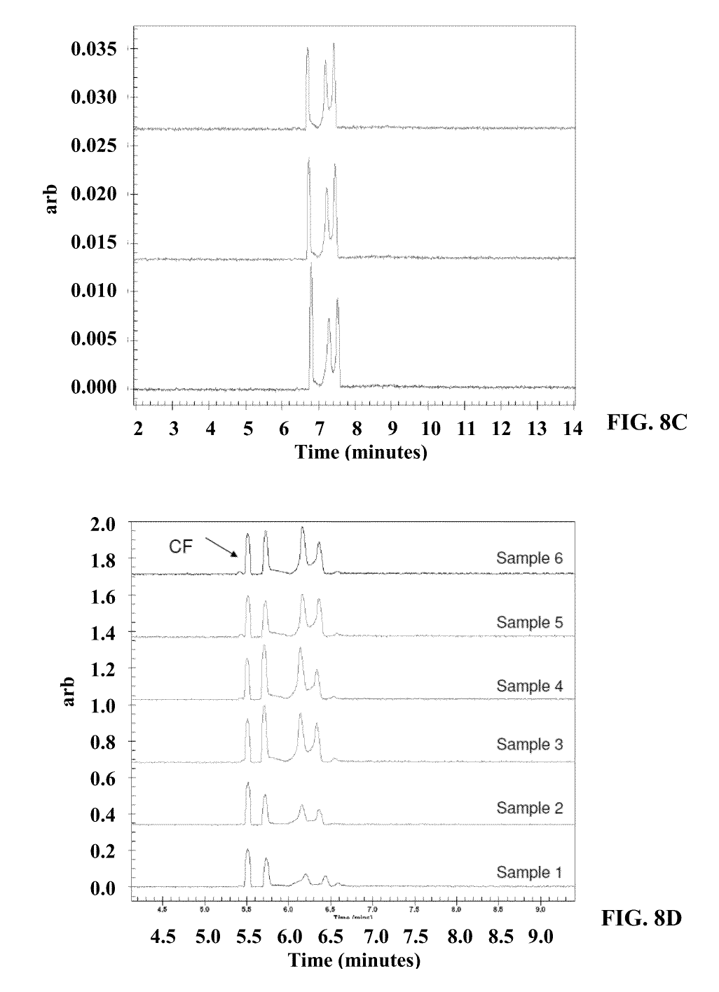

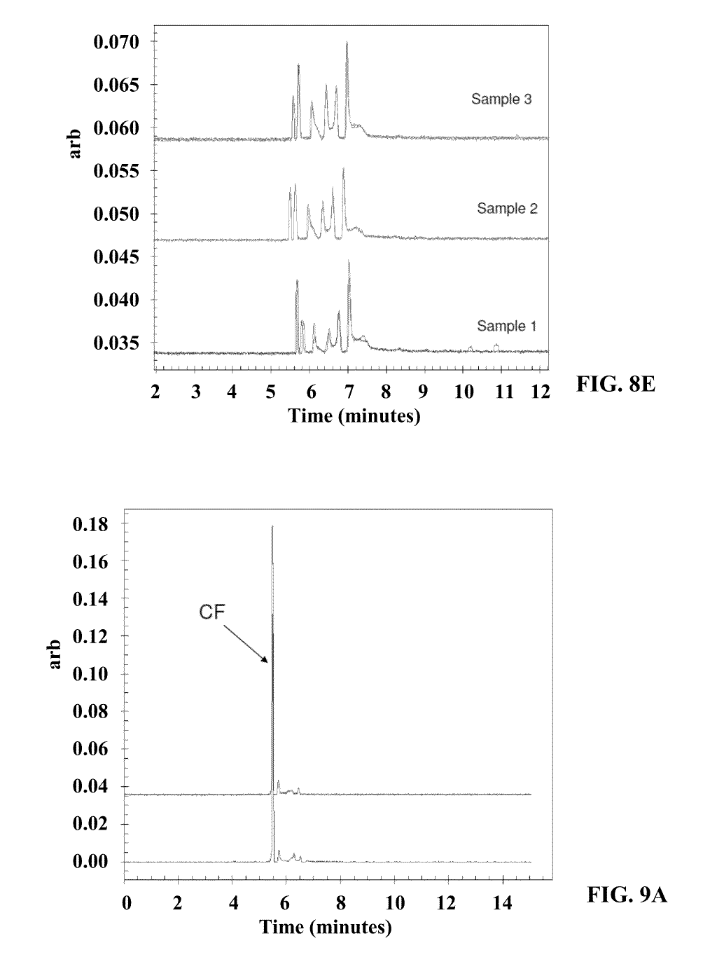

To further evaluate the reproducibility of the system, several samples with known lipoprotein profiles were evaluated. Samples from patients 1, 2, and 6 were previously determined to be Lp(a) positive. Samples from patient 4 were previously determined to be Lp(a) negative. FIG. 8A shows an alignment of the lipid profiles from patient 6 (top) and patient 4 (bottom). The arrows in FIG. 8B indicate the possible location of a Lp(a) peak in samples 1, 2, and 6.

### Example 5

### Quantification of HDL and LDL

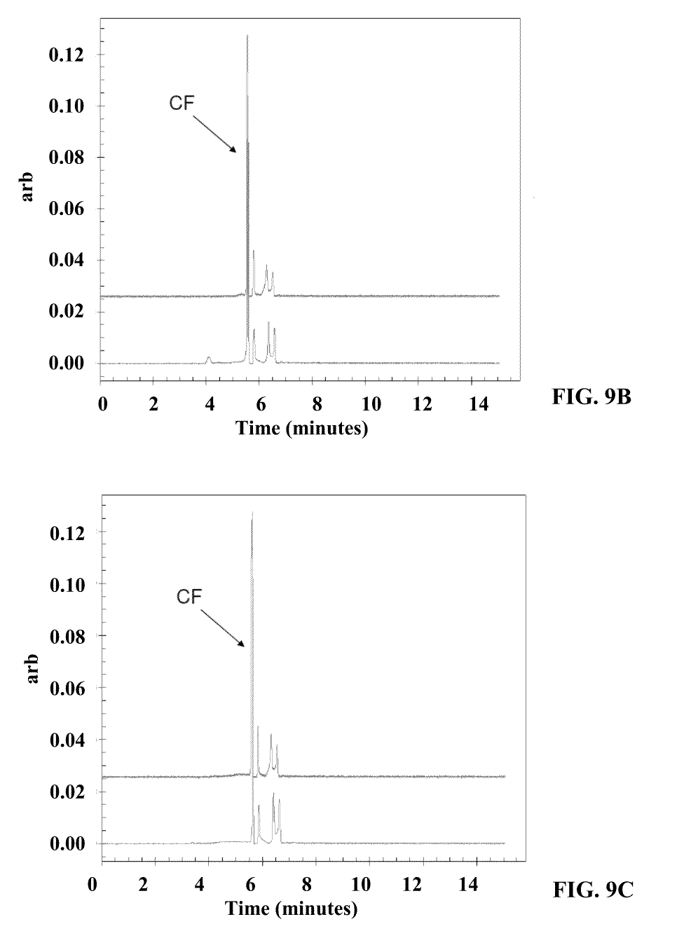

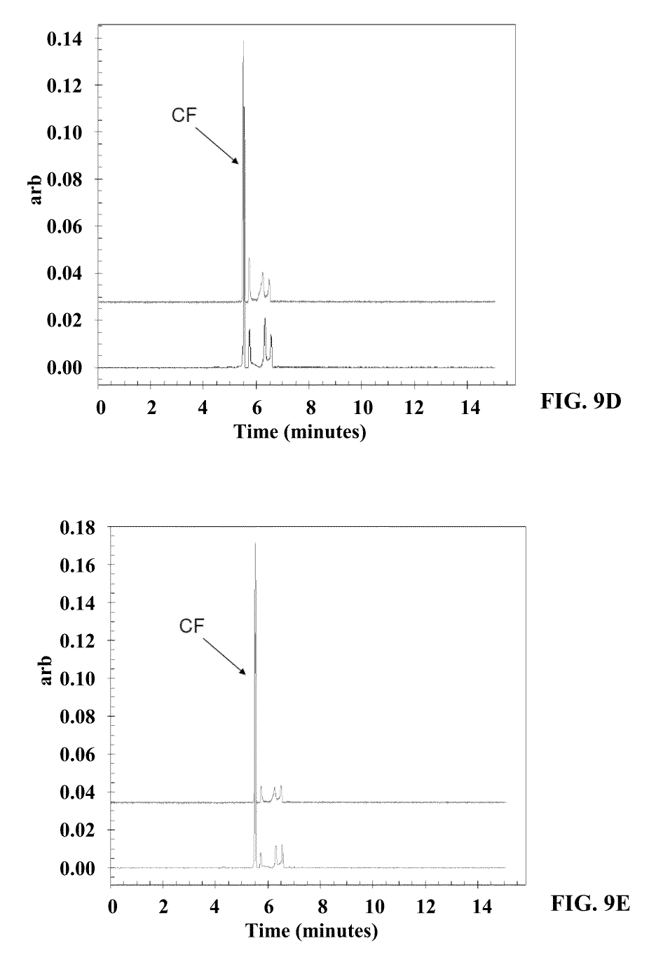

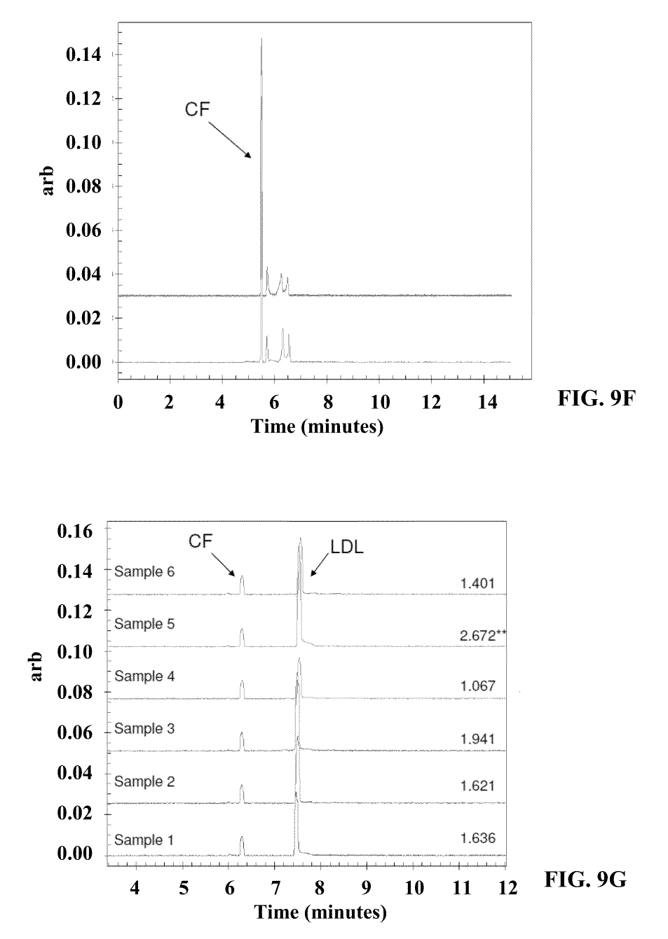

To determine the amounts of HDL and LDL in each of the six patient samples, samples were compared and relative quantities were calculated. Individual electropherograms corresponding to samples 1-6 are shown in FIGS. 9A-9F. The electropherograms were aligned and normalized around the CF peak, which accounts for any fluctuations in the injection (FIG. 9G). The relative amount of HDL in each of the 6 patient samples is shown in Table 1 below. It is possible that sample 4 in FIG. 9G may have had a 2×CF spike. Accordingly, the corrected area would be half of that indicated on the graph for this sample (FIG. 9D).

TABLE 1

| | | Corrected | |
|---|---|---|---|
| | | Peak | % |
| Sample | Peak | Area | Area |
| HDL | CF | 0.764 | 83.15 |
| | HDL A | 0.0638 | 6.95 |
| | HDL B | 0.593 | 6.46 |
| | HDL C | 0.0224 | 2.44 |
| | HDL D | 0.0926 | 1.01 |
| | Total HDL | 0.16 | |
| HDL 2 | CF | 0.614 | 64.3 |
| | HDL A | 0.112 | 11.69 |
| | HDL B | 0.128 | 13.44 |
| | HDL C | 0.0953 | 9.98 |
| | HDL D | 0.00575 | 0.6 |
| | Total HDL | 0.34 | |
| HDL 3 | CF | 0.546 | 57.91 |
| | HDL A | 0.121 | 12.8 |
| | HDL B | 0.14 | 14.81 |
| | HDL C | 0.131 | 13.85 |
| | HDL D | 0.0059 | 0.63 |
| | Total HDL | 0.4 | |
| HDL 4 | CF | 0.623 | 59.43 |
| | HDL A | 0.136 | 12.99 |
| | HDL B | 0.171 | 16.26 |
| | HDL C | 0.113 | 10.75 |
| | HDL D | 0.0059 | 0.56 |
| | Total HDL | 0.43 | |
| HDL 5 | CF | 0.671 | 72.23 |
| | HDL A | 0.0716 | 7.71 |
| | HDL B | 0.0885 | 9.53 |
| | HDL C | 0.0927 | 9.98 |
| | HDL D | 0.00517 | 0.56 |
| | Total HDL | 0.26 | |
| HDL 6 | CF | 0.645 | 66.16 |
| | HDL A | 0.0989 | 10.14 |
| | HDL B | 0.131 | 13.47 |
| | HDL C | 0.0959 | 9.83 |
| | HDL D | 0.00392 | 0.4 |
| | Total HDL | 0.33 | |

1. A method for determining the molar concentration and/or particle number of a lipoprotein or lipid particle present in a biological sample, comprising:
(a) contacting the biological sample with a non-specific lipophilic dye under conditions suitable for the non-specific lipophilic dye to bind to the lipoprotein, or a lipid particle thereof, to form a lipophilic dye-labeled lipoprotein, wherein the biological sample comprises a signal-producing lipoprotein standard;
(b) subjecting the dye-labeled lipoprotein to a capillary isotachophoresis laser-induced fluorescence (CE-ITP-LIF) system;
(c) detecting and comparing signals produced by the non-specific lipophilic dye and the signal-producing lipoprotein standard; and
(d) quantifying, based on said detecting and comparing, the molar concentration and/or particle number of the lipoprotein or lipid particle in the sample, wherein the detected signals are proportional to the molar concentration and/or particle number of the lipoprotein or lipid particle in the sample.

2. The method of claim 1, wherein the CE-ITP-LIF system separates the components of the sample from one another along a common capillary.

3. The method of claim 1, wherein the method is a method for determining the molar concentration and/or particle number of a lipoprotein and the lipoprotein is selected from the group consisting of very low-density lipoprotein (VLDL), low-density lipoprotein (LDL), intermediate-density lipoprotein (IDL), high-density lipoprotein (HDL), chylomicron, lipoprotein X, lipoprotein(a), and subforms and mixtures thereof.

4. The method of claim 1, wherein the CE-ITP-LIF system is a multiplex capillary isotachophoresis laser induced fluorescence (MPCE-ITP-LIF).

5. The method of claim 1, wherein the signal-producing lipoprotein standard comprises a standard lipoprotein or lipid particle with a known concentration, a known radius, a known lipid concentration, a known lipid distribution, or a combination thereof.

6. The method of claim 1, wherein the signal produced by the signal-producing lipoprotein standard is measured and compared with the signal produced from the lipophilic dye-labeled lipoprotein or lipid particle and the molar concentration and/or particle number of the lipoprotein or lipid particle is determined based on the following formula:
$$PN = [ PL ] \times \frac{63662 {Å}^{2} nmol dL}{( L ) ( mg ) ( \% PL ) ( {r}^{2} )} ;$$
where PN is the particle number of the lipoprotein in (nmol/L); [PL] is phospholipid concentration of the lipoprotein in(mg/dL); r² is the radius of the lipoprotein in (A) squared; and % PL is the percent phospholipid in the surface area of the lipoprotein.

7. The method of claim 6, wherein the lipoprotein and/or lipid particle [PL], r², and % PL values are proportional to the signal-producing standard [PL], r², and % PL values.

8. A method of assessing a health risk in a subject, comprising:
(i) determining the particle number and/or molar concentration of a lipoprotein or lipid particle in a biological sample from the subject; and
(ii) assessing the health risk of the subject based on the particle number and/or molar concentration of the lipoprotein or lipid particle; wherein the particle number and/or molar concentration of the lipoprotein or lipid particle is determined by the steps of:
(a) contacting the biological sample with a non-specific lipophilic dye under conditions suitable for the non-specific lipophilic dye to bind to the lipoprotein, or a lipid particle, thereof to form a lipophilic dye-labeled lipoprotein, wherein the biological sample comprises a known concentration of a signal-producing lipoprotein standard;
(b) subjecting the lipophilic dye-labeled lipoprotein to a capillary isotachophoresis laser-induced fluorescence (CE-ITP-LIF) system;
(c) detecting and comparing signals produced by the non-specific lipophilic dye and the signal-producing lipoprotein standard; and
(d) quantifying, based on said detecting and comparing, the particle number and/or molar concentration of the of the lipoprotein or lipid particle in the sample, wherein the detected signals are proportional to the particle number and/or molar concentration of the lipoprotein or lipid particle in the sample.

9. The method of claim 8, wherein the method is a method for determining the molar concentration and/or particle number of a lipoprotein and the lipoprotein is selected from the group consisting of very low-density lipoprotein (VLDL), low-density lipoprotein (LDL), intermediate-density lipoprotein (IDL), high-density lipoprotein (HDL), chylomicron, lipoprotein X, lipoprotein(a), and subforms and mixtures thereof.

10. The method of claim 8, wherein the signal-producing lipoprotein standard comprises a standard lipoprotein or lipid particle with a known concentration, a known radius, a known lipid concentration, a known lipid distribution, or a combination thereof.

11. The method of claim 10, wherein the signal produced by the signal-producing lipoprotein standard is measured and compared with the signal produced from the lipophilic dye-labeled lipoprotein or lipid particle and the molar concentration and/or particle number of the lipoprotein or lipid particle is determined based on the following formula:
$$PN = [ PL ] \times \frac{63662 {Å}^{2} nmol dL}{( L ) ( mg ) ( \% PL ) ( {r}^{2} )} ;$$
where PN is the particle number of the lipoprotein in (nmol/L); [PL] is phospholipid concentration of the lipoprotein in (mg/dL); r² is the radius of the lipoprotein in (A) squared; and % PL is the percent phospholipid in the surface area of the lipoprotein.

12. The method of claim 8, wherein the subject is assigned to one of a low, moderate, or high health risk categories based on the particle number and/or molar concentration of the lipoprotein.

13. The method of claim 8, wherein the health risk is a risk associated with a cardiovascular disorder, a metabolic disorder, or diabetes.

14. The method of claim 12, wherein the method further comprises administering to the subject a therapeutic regimen for reducing the health risk, or modifying an existing therapeutic regimen for the subject for reducing the health risk, based on the health risk category assigned to the subject.

15. The method of claim 14, wherein the therapeutic regimen comprises administering a drug and/or a supplement or the existing therapeutic regimen comprises administering a modified dose of a drug and/or a supplement.

16. The method of claim 15, wherein the drug is selected from the group consisting of niacin, an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, an angiotensin system inhibitor, and combinations thereof.

17. The method of claim 15, wherein the drug is selected from the group consisting of niacin, statin, ezetimibe, fenofibrate, estrogen, raloxifene and combinations thereof.

18. The method of claim 16, wherein the selected therapeutic regimen involves giving recommendations on making or maintaining lifestyle choices based on the results of said health risk determination.

19. The method of claim 18, wherein the lifestyle choices involve changes in diet, changes in exercise, reducing or eliminating smoking, or a combination thereof.

20. The method of claim 1, wherein the biological sample is selected from the group consisting of blood, plasma, urine and saliva.

21. The method of claim 1, wherein the non-specific lipophilic dye is selected from the group consisting of NDB-ceramide, ADIFAB fatty acid indicators, phospholipids with BODIPY dye-labeled acyl chains, phospholipid with DPH-labeled acyl chains, phospholipids with NBD-labeled acyl chains, phospholipids with pyrene-labeled acyl chains, phospholipids with a fluorescent or biotinylated head groups, LipidTOX phospholipid, neutral lipid stains and combinations thereof.

22. The method of claim 8, wherein the biological sample is selected from the group consisting of blood, plasma, urine and saliva.

23. The method of claim 8, wherein the non-specific lipophilic dye is selected from the group consisting of NDB-ceramide, ADIFAB fatty acid indicators, phospholipids with BODIPY dye-labeled acyl chains, phospholipid with DPH-labeled acyl chains, phospholipids with NBD-labeled acyl chains, phospholipids with pyrene-labeled acyl chains, phospholipids with a fluorescent or biotinylated head groups, LipidTOX phospholipid, neutral lipid stains and combinations thereof.
